(19) United States
(12) Patent Application Publication
(10) Pub. No.: US 2026/0331221 A1
(43) Pub. Date: Oct. 22, 2026

(54) METHODS AND COMPOSITIONS FOR CANCER SUBTYPING USING INTEGRATED GENOMIC AND EPIGENOMIC SIGNATURES

(71) Applicant: GUARDANT HEALTH, INC., Palo Alto, CA (US)

(72) Inventor: Aaron Isaac HARDIN, San Francisco, CA (US)

(21) Appl. No.: 19/648,950

(22) Filed: Apr. 15, 2026

Related U.S. Application Data
(60) Provisional application No. 63/789,623, filed on Apr. 16, 2025.
(60) Provisional application No. 63/840,150, filed on Jul. 8, 2025.

(51) Int. Cl. G16B 20/20; G16B 40/30

(52) U.S. Cl. CPC G16B 20/20; G16B 40/30

(57) ABSTRACT

Disclosed herein are methods, systems, and compositions for stratifying subjects into clinically relevant subgroups using integrated multi-omic data. The methods comprise receiving clinical data, genomic data, and epigenomic data associated with a subject; generating derived features including dimension-reduced epigenomic representations; applying ensemble clustering techniques to assign the subject to a subgroup; and associating the subgroup with a clinical outcome. In certain embodiments, the methods further comprise identifying prognostic biomarkers and generating risk scores using survival models. The disclosed approaches enable improved disease subtyping, prognosis, and therapeutic decision-making across a variety of diseases, including cancer.

### CROSS-REFERENCE TO RELATED APPLICATIONS

This application claims the benefit of U.S. Provisional Application No. 63/789,623, filed Apr. 16, 2015, and 63/840,150, filed Jul. 8, 2015, wherein each application is incorporated herein by reference in its entirety.

### FIELD OF THE INVENTION

The present disclosure relates to computational biology, bioinformatics, and precision medicine, and more particularly to methods and systems for disease subtyping and prognosis using integrated multi-modal biological data including genomic and epigenomic information.

### BACKGROUND

Disease prognosis and treatment response often vary significantly across patients due to complex interactions among clinical, genetic, and epigenetic factors. While advances in sequencing technologies have enabled the generation of large-scale genomic and epigenomic datasets, existing approaches typically analyze such data modalities in isolation, limiting their predictive utility. Conventional approach do not account for the observation that further differences may be present in the normal host tissue, or they may be acquired by cancer cells during transformation. Such differences may affect diverse components of treatment response, including: a drug's pharmacokinetics (e.g., metabolism or transport) or pharmacodynamics (e.g., a target or modulating enzyme); host tissue sensitivity to radiation; the sensitivity of malignant cells to cytotoxic agents, including drugs and radiation; and the ability of malignant cells to invade and metastasize.

Additionally, one of the reasons cancer is so difficult to treat is that current testing methods often do not help doctors match specific cancers with effective drug treatments. Moreover, the disease state itself can be a moving target—cancer cells are constantly changing and mutating. Although cancer tumors continually shed their unique genomic material into the bloodstream, unfortunately, these telltale genomic “signals” are so weak that current genomic analysis technologies, including next-generation sequencing, can only detect such signals sporadically or in patients with terminally high tumor burden. The main reason for this is that such technologies are plagued by error rates and bias that can be orders of magnitude higher than what is required to reliably detect de novo genomic alterations associated with cancer. Thus, improved systems and methods for determining effective treatments for cancer are needed.

There remains a need for improved computational frameworks that integrate heterogeneous biological and clinical data to identify clinically meaningful disease subtypes, predict outcomes, and guide therapeutic strategies.

### SUMMARY OF THE INVENTION

Described herein is a computer-implemented method including receiving, for a plurality of subjects, at least two of: clinical data, genomic data, epigenomic data, transcriptomic data, and proteomic data, generating a plurality of features including one or more derived epigenomic features obtained via dimensionality reduction, applying a plurality of clustering algorithms to the features to generate candidate cluster assignments, generating a consensus clustering based on the candidate cluster assignments, assigning each subject to a cluster; and associating the cluster with a clinical outcome. In various embodiments, the method includes epigenomic data including DNA methylation data. In various embodiments, the method includes derived epigenomic features that comprise principal components. In various embodiments, the method includes dimensionality reduction that includes principal component analysis. In various embodiments, the method includes dimensionality reduction that includes a machine learning model. In various embodiments, the genomic data comprise mutation data. In various embodiments, the mutation data comprise mutations in one or more cancer-associated genes. In various embodiments, the comprise one or more of: PAX6, SOX9, OTX2, WT1, TBX20, KRAS, STK11, and KEAP1. In various embodiments, the algorithms comprise at least two different clustering methods. In various embodiments, the clustering methods comprise k-means and hierarchical clustering. In various embodiments, the clustering methods further comprise density-based clustering. In various embodiments, generating the consensus clustering includes constructing a matrix, optionally including co-association. In various embodiments, the method includes evaluating cluster stability. In various embodiments, the clinical outcome includes one or more of: progression-free survival, overall survival, progression, recurrence, adverse event, and time to next treatment.

In various embodiments, the method includes fitting a survival model. In various embodiments, the survival model includes a Cox proportional hazards model. In various embodiments, the method includes identifying one or more prognostic biomarkers. In various embodiments, the disease includes cancer. In various embodiments, the cancer includes lung cancer. In various embodiments, the epigenomic features correspond to methylation patterns associated with developmental genes. In various embodiments, the genes comprise one or more of: PAX6, SOX9, OTX2, WT1, TBX20, KRAS, STK11, and KEAP1. In various embodiments, the features further comprise clinical treatment history. In various embodiments, the method includes prediction of disease. In various embodiments, the method includes selection of a treatment. In various embodiments, the system is implemented in a cloud computing environment.

In various embodiments, the epigenomic data are derived from a blood sample. In various embodiments, the epigenomic data are derived from a tissue sample. In various embodiments, the features further comprise genomic, transcriptomic or proteomic data. In various embodiments, assigning the subject to the cluster includes assigning a new subject using a trained model. In various embodiments, the therapy includes a targeted therapy selected based on the cluster.

Described herein is a system including one or more processors and memory storing instructions that, when executed, cause the system to perform the aforementioned methods. In various embodiments, this includes a computer-implemented method including receiving, for a plurality of subjects, at least two of: clinical data, genomic data, epigenomic data, transcriptomic data, and proteomic data, generating a plurality of features including one or more derived epigenomic features obtained via dimensionality reduction, applying a plurality of clustering algorithms to the features to generate candidate cluster assignments, generating a consensus clustering based on the candidate cluster assignments, assigning each subject to a cluster; and associating the cluster with a clinical outcome. In various embodiments, the system further including an interface for receiving biological data.

Further described herein is a non-transitory computer-readable medium storing instructions that, when executed, cause a system to perform the aforementioned methods. In various embodiments, this includes a computer-implemented method including receiving, for a plurality of subjects, at least two of: clinical data, genomic data, epigenomic data, transcriptomic data, and proteomic data, generating a plurality of features including one or more derived epigenomic features obtained via dimensionality reduction, applying a plurality of clustering algorithms to the features to generate candidate cluster assignments, generating a consensus clustering based on the candidate cluster assignments, assigning each subject to a cluster; and associating the cluster with a clinical outcome. the instructions further cause identification of one or more prognostic biomarkers. In various embodiments, the method includes assigning the subject to a cluster using the method of claim 1 and selecting a therapy based on the cluster assignment.

### BRIEF DESCRIPTION OF THE DRAWINGS

FIG. 1. Illustrative combined genomic-epigenomic detection platform workflow.

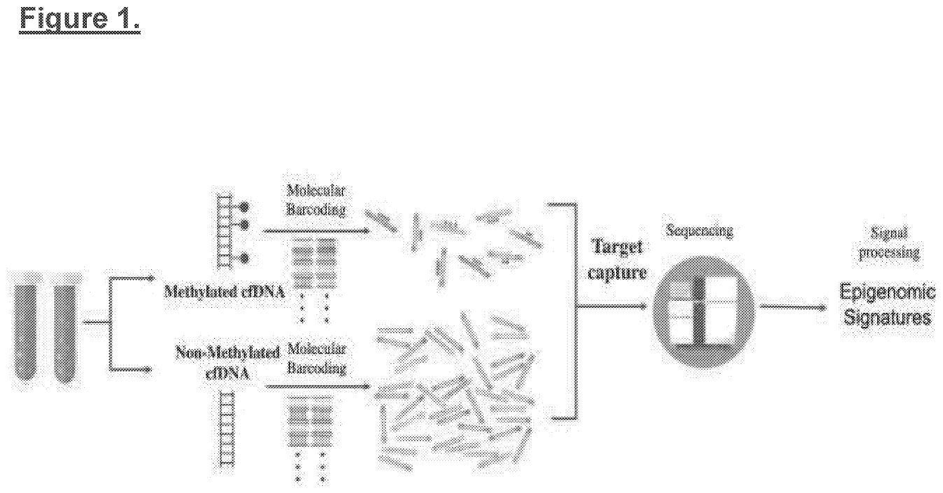

FIG. 2. Consensus matrix stability plot displaying the agreement in patient cluster assignments across multiple clustering methods (K-means, hierarchical with average, Ward, and complete linkage, DBSCAN, HDBSCAN, and Gaussian mixture modeling). Each entry represents the proportion of methods in which a given pair of patients was assigned to the same cluster, ranging from 0 (no agreement; light color) to 1 (perfect agreement; dark color). Lack of clear block-diagonal patterns suggests overall limited cluster stability, indicating variability or uncertainty in patient subgrouping across methods.

FIG. 3. Heatmap depicting binary mutation indicators for the most frequently mutated genes and principal components (PCs) derived from methylation data, organized by patient clusters determined via consensus clustering. Rows represent individual patients sorted by cluster assignment; columns represent selected mutation indicators (prefixed by “gene_”) and methylation principal components (“methPC”). The color intensity corresponds to standardized feature values (red indicating positive/higher values, blue negative/lower values). The plot enables visual identification of mutation and methylation patterns distinctive to each cluster, although the overall predominance of lighter shading suggests limited strong differentiation among clusters for these features.

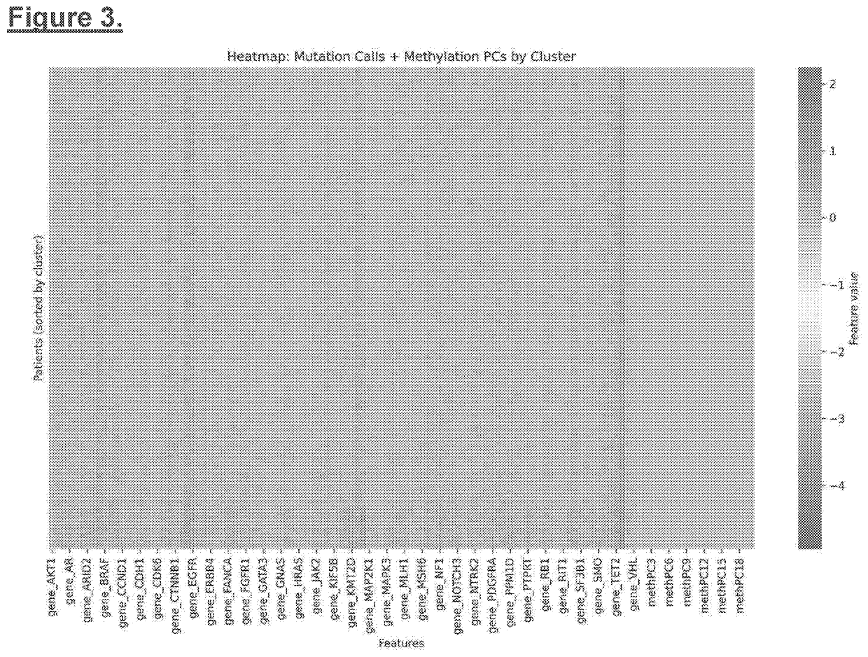

FIG. 4. Kaplan-Meier survival curves illustrating overall survival differences among patient clusters derived from integrative consensus clustering. clustering on clinical, genomic, and methylation data. Shaded areas represent 95% confidence intervals. Cluster 1 (blue line) represents the largest subgroup (N=3815), exhibiting the poorest survival. Cluster 2 (orange line, N=277) and Cluster 3 (green line, N=85) display improved survival, although Cluster 3's small size may reflect an outlier subgroup.

FIG. 5. Forest plot depicting the top prognostic features identified by the penalized Cox model. Hazard ratios (HRs) with corresponding 95% confidence intervals are shown for key somatic mutations and methylation principal component 5 (methPC5). HR values greater than 1 indicate increased risk of mortality, whereas values less than 1 indicate reduced risk. Mutations in STK11, KEAP1, and KRAS are associated with significantly worse prognosis, whereas higher scores on methPC5 indicate substantially improved outcomes.

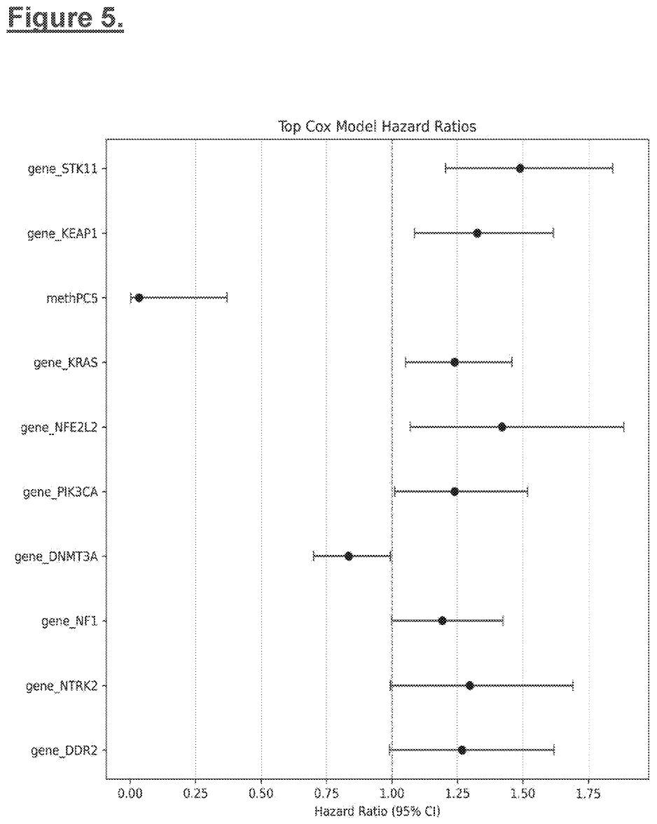

### DETAILED DESCRIPTION

Described herein is a comprehensive framework for integrating multiple classes of biological and clinical data in order to identify disease subtypes and predict clinical outcomes. The Inventors have recognized that clinically meaningful heterogeneity in diseases such as cancer arises from the combined effects of clinical variables, genomic alterations, and epigenetic regulation. By jointly analyzing these data modalities, the disclosed methods capture underlying biological structure that is not detectable through single-modality analyses.

For example, methods and compositions are described herein, where data associated with a plurality of subjects are obtained from one or more sources. Such data may include clinical information, such as treatment history, therapeutic regimens, disease stage, and demographic variables. In addition, genomic data may be obtained from sequencing assays and may include, for example, somatic mutations, copy number variations, structural variants, or other genomic features. Epigenomic data may also be obtained, including measurements of DNA methylation across genomic regions. These data may be derived from tissue samples, blood samples, or other biological specimens, and may be generated using any suitable assay or sequencing technology.

Further described is a scalable computational framework for integrating multi-modal biological data to identify disease subtypes associated with clinically meaningful outcomes. In various embodiments, the disclosed methods integrate clinical, genomic, and epigenomic data to generate composite representations that capture underlying biological heterogeneity.

In some embodiments, data are obtained from one or more sources including clinical records (e.g., treatment history, demographics), genomic sequencing data (e.g., somatic mutations, copy number alterations), and/or epigenomic data (e.g., DNA methylation profiles). In various embodiments, the data may be derived from tissue samples, blood samples, or other biological specimens.

Following acquisition, the data are processed to generate features suitable for downstream analysis. Clinical variables may be encoded as categorical or continuous variables, while genomic data may be represented as binary indicators reflecting the presence or absence of alterations in particular genes or genomic regions. Epigenomic data, which may comprise high-dimensional measurements, are transformed to generate derived features that capture latent structure within the data. In some embodiments, dimensionality reduction techniques such as principal component analysis are applied to methylation data to generate a set of principal components that summarize variation across samples. In other embodiments, alternative techniques such as matrix factorization methods or machine learning models are employed to generate latent representations.

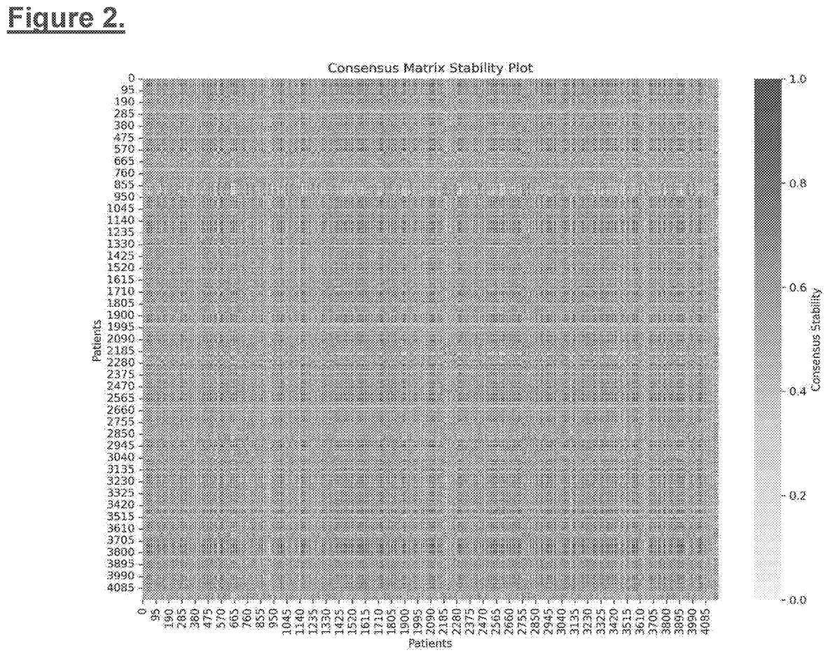

Further described herein are clustering algorithms that serve to capture distinct aspects of structure within the data. Accordingly, in certain embodiments, multiple clustering methods are applied to the generated features. These methods may include partition-based approaches, hierarchical clustering techniques, density-based clustering, and probabilistic mixture models. Each clustering method produces a candidate assignment of subjects into clusters. Various unsupervised learning techniques may be employed to partition a set of subjects into subgroups, including, for example, partition-based algorithms, hierarchical clustering approaches using different linkage criteria, density-based methods, probabilistic or statistical mixture models, or other machine learning-based clustering techniques. In some embodiments, each technique independently generates a candidate assignment of subjects to one or more clusters based on underlying feature representations. These candidate assignments may be generated using different parameterizations, initialization schemes, distance metrics, or feature subsets, thereby providing multiple alternative views of the underlying data structure. In certain implementations, the plurality of candidate clusterings may be further compared, aggregated, or refined to produce a consensus or optimized clustering, although in other embodiments individual clustering outputs may be selected or weighted based on predefined criteria or performance metrics. These assignments are then combined to generate a consensus clustering, for example by constructing a co-association matrix that reflects the frequency with which pairs of subjects are assigned to the same cluster across methods. As illustrated in FIG. 2, such consensus approaches enable assessment of clustering stability and identification of robust subgroup structures.

For example, a stability representation may be generated to evaluate the consistency of subject clustering across multiple clustering approaches. In certain embodiments, clustering assignments for a plurality of subjects are obtained using two or more distinct clustering algorithms, which may include, for example, partition-based methods, hierarchical clustering methods employing different linkage criteria, density-based clustering techniques, probabilistic mixture models, or other unsupervised learning approaches.

A consensus matrix or similar structure may then be constructed to quantify agreement between clustering results. In some implementations, each entry of the matrix corresponds to a pairwise comparison between subjects and represents a measure of co-assignment, such as the frequency, proportion, probability, or weighted score indicating how often the subjects are assigned to the same cluster across the plurality of clustering methods or across multiple runs of a given method. The values may be normalized to a defined range, for example between zero and one, or expressed using alternative scales or similarity metrics.

The consensus matrix may be visualized using a graphical representation, such as a heatmap or other visual encoding, in which varying intensities, colors, or patterns reflect levels of agreement. In certain embodiments, the presence of well-defined block structures within the matrix may indicate stable subgrouping of subjects, whereas diffuse or non-distinct patterns may indicate reduced clustering stability, ambiguity in subgroup boundaries, or heterogeneity within the dataset.

In some embodiments, measures of clustering stability may be further quantified using statistical or computational metrics, including but not limited to consensus scores, silhouette scores, variation of information, or resampling-based stability indices. Such analyses may be used to select among clustering methods, determine an optimal number of clusters, assess robustness of identified subgroups, or guide downstream analyses.

In alternative implementations, consensus or stability assessments may be performed across different feature sets, data modalities, or preprocessing strategies, thereby enabling evaluation of the sensitivity of clustering results to variations in input data or modeling assumptions. Additionally, stability information may be incorporated into downstream processes, such as weighting cluster assignments, filtering unreliable subgroupings, or informing confidence scores associated with subject classification.

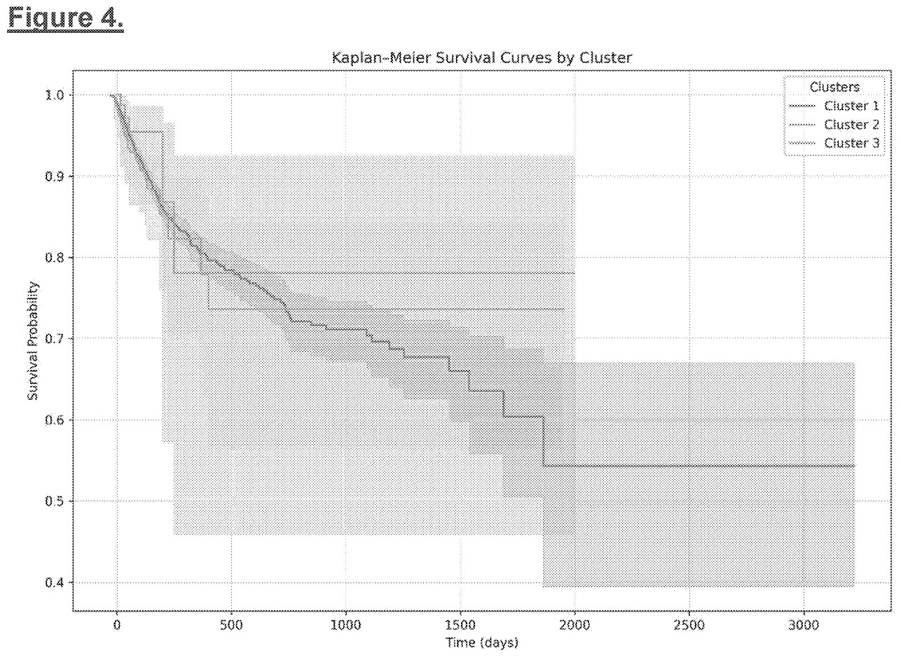

Once cluster assignments are established, the resulting subgroups are evaluated with respect to clinical outcomes. In certain embodiments, survival analysis techniques are applied to assess differences in outcomes between clusters. For example, Kaplan-Meier survival curves may be generated to visualize differences in survival probabilities over time, as shown in FIG. 4. Statistical tests such as log-rank tests may be used to determine whether observed differences are significant. In addition, regression-based survival models, such as Cox proportional hazards models, may be fitted to quantify the contribution of individual features to risk.

Through such modeling, specific genomic and epigenomic features may be identified as prognostic biomarkers. For example, mutations in certain genes may be associated with increased risk, while derived epigenomic features may be associated with protective effects. In particular embodiments, a latent epigenomic feature derived from methylation data may correspond to coordinated methylation changes in genes involved in developmental or differentiation pathways, thereby reflecting underlying biological mechanisms that influence disease progression. These findings demonstrate that integration of epigenomic features provides additional prognostic information beyond genomic data alone.

The disclosed methods may be applied in a variety of clinical contexts. In some embodiments, a subject's data are processed according to the methods described herein, and the subject is assigned to a cluster corresponding to a disease subtype. This subtype assignment may then be used to inform prognosis, guide treatment selection, or identify eligibility for particular therapies. For example, subjects assigned to high-risk clusters may be directed toward more aggressive treatment regimens, while subjects in lower-risk clusters may receive less intensive therapies.

The methods described herein may be implemented using one or more computing systems. Such systems may include processors configured to execute instructions stored in memory, as well as interfaces for receiving input data and providing output results. The systems may be deployed in clinical laboratories, research environments, or cloud-based platforms, and may be integrated with electronic health record systems or laboratory information systems.

Although particular embodiments have been described, it will be appreciated that numerous variations and modifications may be made without departing from the scope of the present disclosure. For example, additional data modalities such as transcriptomic, proteomic, or metabolomic data may be incorporated into the analysis. Similarly, alternative feature engineering, clustering, or modeling techniques may be employed. The disclosed framework is therefore broadly applicable to a wide range of diseases and analytical settings.

More information is found in PCT Pub. Nos. WO 2017062867, WO2023034453, each of which are fully incorporated by reference herein.

Further described herein are methods and compositions related to feature engineering based on the detection platform and data analysis processes. In various embodiments, features may include: clinical variables (e.g., treatment lines, regimens), genomic indicators (e.g., binary mutation presence for selected genes), and/or epigenomic measurements (e.g., methylation levels across genomic regions).

In certain embodiments, epigenomic data are transformed into derived features using dimensionality reduction techniques, including: Principal component analysis (PCA), Non-negative matrix factorization (NMF), and/or autoencoders or other machine learning models. In various embodiments, these derived features capture latent biological signals not readily observable from raw measurements. In various embodiments, the methods and compositions described herein are utilized with any cancer type, non-cancer diseases (e.g., autoimmune, neurological). In various embodiments, additional data modalities may include: transcriptomics, proteomics, and/or metabolomics.

Feature generation, clustering, and modeling techniques may be substituted with equivalent methods without departing from the scope of the invention. For example, when analyzing genomic regions in an individual sample, a computing system can determine a number of sequence representations that correspond to polynucleotide molecules having at least the threshold number of methylated cytosines in the genomic region. The computing system can perform one or more normalization operations using the counts of polynucleotide molecules or sequence reads that correspond to the genomic region and have at least the threshold number of methylated cytosines to generate normalized metrics. To illustrate, the computing system can divide the counts of polynucleotide molecules or reads that correspond to the genomic region and have at least the threshold number of methylated cytosines by the number of molecules or sequencing reads that correspond to a control region, such as a positive control region. In another instance, the computing system can perform the normalized metrics by dividing the counts of polynucleotide molecules or reads that correspond to the genomic region and have at least the threshold number of methylated cytosines by the number of molecules or sequencing reads in a control dataset (i.e., the control dataset includes of tumor not-detected samples) corresponding to the same genomic region and have at least the same threshold number of methylated cytosines.

The normalized metrics can be analyzed with respect to a threshold value. The threshold value can correspond to a given genomic region, such as a given promoter region. In various examples, the threshold value can be different for different promoter regions. In these scenarios, a first promoter region can have a first threshold value and a second promoter region can have a second threshold value. In situations where the normalization metric is at least the threshold value, the computing system can determine that the genomic region has a first methylation status. In scenarios where the normalization metric is less than the threshold value, the computing system can determine that the genomic region has a second methylation status. In one or more illustrative examples, the first methylation status can be labeled as “methylated” and the second methylation status can be labeled as “not methylated.”

The threshold value for a given genomic region can be determined based on training data obtained from samples of individuals in which cancer is not detected. In one or more examples, sequence representations obtained from the training samples can be analyzed to determine a z-score with respect to the number of polynucleotide molecules that correspond to the genomic region and that have at least the threshold amount of methylated cytosines. In one or more illustrative examples, the threshold value for a promoter region that is used to determine the normalization metrics for the promoter region can be derived from the z-score calculated based on the training samples with respect to the promoter region.

In various embodiments, sequencing data can be analyzed by the computing system to determine indicators of the presence of cancer without training specific models. In one or more examples, the computing system can determine a tumor fraction value based on sequencing data generated from one or more samples obtained from a single subject in which it is unknown whether or not cancer is present in the subject. In various examples, the plurality of samples obtained from a given subject can be produced by performing a number of titrations on a single sample. In one or more additional examples, a first sample can be obtained from a subject prior to or at onset of at least one administration of a treatment or a procedure related to cancer and one or more second samples can be obtained from the subject after at least one of administration of a treatment or a procedure related to cancer. In one or more illustrative examples, the one or more second samples can be obtained at least one week, at least two weeks, at least three weeks, at least four weeks, at least five weeks, at least six weeks, at least eight weeks, or at least ten weeks after administration of the treatment or procedure. In at least some examples, first sample and the second sample can be derived from at least one of a bodily fluid obtained from the subject or tissue obtained from the subject.

In one or more examples, one or more samples can be obtained from a given subject. The sequencing data generated from the one or more samples can be analyzed by the computing system to determine quantitative measures for a number of classification regions. In one or more examples, the quantitative measures can correspond to an amount of sequence representations that have at least a threshold amount of overlap with one or more classification regions. In one or more additional examples, the quantitative measures can correspond to sequence representations having at least a threshold amount of methylated cytosines in CpG regions having at least a threshold amount of CG content. In various examples, the indication of cancer status in the subject can include tumor fraction. In one or more additional examples, the indication of cancer status in the subject can include mutant allele fraction. In at least some examples, the quantitative measures can correspond to a number of sequencing reads that correspond to a given classification region in relation to a total number of sequencing reads across a plurality of positive control regions. In one or more further examples, the indicators of cancer status can be used to determine an output that corresponds to cancer status or not being present in a given individual in response to analyzing the one or more indicators of cancer status with respect to one or more thresholds. In one or more illustrative examples, tumor fraction determined from one or more samples obtained from a subject can be analyzed with respect to one or more thresholds. In instances where tumor fraction is greater than a threshold level, the computing system can determine that the probability of cancer status in the subject is at least 80%, at least 85%, at least 90%, at least 95%, or at least 99%. Further, in situations where multiple samples are obtained from a subject, first quantitative measures generated from a first sample obtained from the subject can be analyzed with respect to second quantitative measures generated from a second sample obtained from the subject. In at least some examples, differences between the first quantitative measures and the second quantitative measures can be analyzed to determine an indication of treatment response in the subject.

The quantitative measures used to determine an indication of cancer status in a subject can be determined by analyzing quantitative measures of a subset of classification regions. In at least some examples, the subset of classification regions can be different for different subjects. In one or more illustrative examples, values of quantitative measures for a number of classification regions can be analyzed with respect to one another and ranked according to the magnitude of the value of the quantitative measures. In various examples, the classification regions for a given sample can be ranked in descending order from the one or more classification regions having the greatest value of a quantitative measure to the one or more classification region having the least value of the quantitative measure.

In various examples, after ranking the quantitative measures of the classification regions, quantitative measures that correspond to a group of the classification regions can be removed before determining the indication of cancer status in the subject. For example, the group of classification regions that are not used to determine the indication of cancer status in the subject can include the 1% of classification regions having the greatest quantitative measure values, the 2% of classification regions having the greatest quantitative measure values, 3% of classification regions having the greatest quantitative measure values, 4% of classification regions having the greatest quantitative measure values, 5% of classification regions having the greatest quantitative measure values, or the 6% of classification regions having the greatest quantitative measure values. In at least some examples, a number of classification regions having relatively high quantitative measure values can be excluded from the group of classification regions used to determine the indication of cancer status in the subject because, in at least some cases, classification regions corresponding to quantitative measure values at or near the top of the ranked list can have non-tumor origins and/or be related to sequencing artifacts. Thus, by removing the quantitative measures that correspond to these classification regions from the analysis used to determine the indication of cancer status in the subject, the accuracy with which the indication of cancer status in the subject can increase.

In one or more examples, after determining the group of classification regions to be used to determine an indication of cancer status in the subject, a subset of classification regions of the group can then be determined by identifying at least 10 classification regions of the group, at least 25 classification regions of the group, at least 50 classification regions of the group, at least 75 classification regions of the group, at least 100 classification regions of the group, at least 150 classification regions of the group, at least 200 classification regions of the group, at least 250 classification regions of the group, at least 300 classification regions of the group, at least 350 classification regions of the group, at least 400 classification regions of the group, at least 450 classification regions of the group, or at least 500 classification regions of the group having the greatest values for the respective quantitative measure.

In at least some examples, one or more statistical measures, such as at least one of mean, median, or mode, can be applied to the quantitative measures of the subset of the classification regions of the group to generate an initial indication of cancer status in the subject. In various examples, the initial indication of cancer can be modified according to a scaling factor. The scaling factor can be applied to the initial indication of cancer status in the subject because, in at least some scenarios, the positive control regions can have different amounts of methylated CpGs. For example, at least a portion of the positive control regions can have fully methylated CpGs while other positive control regions may not be fully methylated. Additionally, in various situations, some classification regions can correspond to a high value of an indication of cancer status in subjects, such as 90% tumor fraction, 95% tumor fraction, 99% tumor fraction, or 100% tumor fraction, but nucleic acid molecules that correspond to these classification regions may not be fully methylated. To account for these cases, the scaling factor can be applied to the initial indication of cancer status in the subject to provide a more accurate determination of the indication. In one or more illustrative examples, the scaling factor can be determined by analyzing indications of cancer status in subjects determined using one or more techniques described herein in relation to additional data that corresponds to additional indications of cancer status in subjects, such as validation data or other techniques that generate data orthogonal to the indications of tumors being present in subjects described herein.

In various examples, the classification regions used to determine the quantitative measures can correspond to classification regions that correspond to one or more portions of differentially methylated regions. In one or more examples, the differentially methylated regions can include promoter regions that correspond to one or more classifications of cancer. For example, the classification regions can be determined by analyzing a number of sequencing representations across a differentially methylated region. In these scenarios, one or more portions of the differentially methylated regions that overlap with at least at threshold number of sequencing representations can be included in the classification regions. In one or more examples, the quantitative measures of the one or more portions of the differentially methylated regions can be determined based on the molecule count distribution of the differentially methylated region. For example, the quantitative measures can be determined based on the molecule count within one or more peaks of the molecule distribution of the differentially methylated region. To illustrate, in various examples, the distribution of molecules across a differentially methylated region can indicate one or more peaks where greater amounts of molecules overlap with one or more subregions within the differentially methylated region. In various examples, the one or more genomic regions that correspond to the one or more subregions of the differentially methylated regions that correspond to the highest amounts of sequence representations for a sample can be defined as classification regions. In at least some examples, the distribution of sequence representations can have a peak that corresponds to a subregion of the differentially methylated region having a higher number of sequence representations than other subregions of the differentially methylated region. In these scenarios, the subregion can be identified as a classification region. By determining subregions of at least a portion of the differentially methylated regions used to determine the indication of cancer status in the subject, the amount of computing resources and memory resources used to determine the indication of cancer status in the subject can be decreased.

To illustrate, a classification region can include one or more portions of a differentially methylated region in which at least 50% of the sequencing representations obtained from a sample overlap, at least 55% of the sequencing representations obtained from a sample overlap, at least 60% of the sequencing representations obtained from a sample overlap, at least 65% of the sequencing representations obtained from a sample overlap, at least 70% of the sequencing representations obtained from a sample overlap, at least 75% of the sequencing representations obtained from a sample overlap, at least 80% of the sequencing representations obtained from a sample overlap, at least 85% of the sequencing representations obtained from a sample overlap, at least 90% of the sequencing representations obtained from a sample overlap, at least 95% of the sequencing representations obtained from a sample overlap, or at least 99% of the sequencing representations obtained from a sample overlap. In one or more illustrative examples, the one or more portions of the differentially methylated region that comprise a classification region can be contiguous with respect to a reference sequence. Further information is found in PCT Pub. No. WO 2024/211717, which is fully incorporated by reference herein.

In various embodiments, the method includes clustering and subtype identification. In some embodiments, multiple clustering algorithms are applied, including. K-means clustering, hierarchical clustering (e.g., Ward linkage, average linkage), density-based clustering (e.g., DBSCAN, HDBSCAN), Gaussian mixture models.

In various embodiments, cluster assignments from multiple methods are combined to generate a consensus clustering, for example using: co-association matrices, voting schemes and/or stability analysis. As illustrated in FIG. 2, consensus clustering may reveal stable or unstable subgroup structures.

In various embodiments, methods and compositions described herein are utilized to determination association with clinical outcomes, subjects assigned to clusters are evaluated with respect to clinical outcomes, including: overall survival, progression-free survival, treatment response. In various embodiments, statistical methods may include: Kaplan-Meier survival analysis (see FIG. 4), Log-rank tests and/or Cox proportional hazards modeling.

In various embodiments, clusters exhibiting statistically significant differences in outcomes may define clinically relevant subtypes. Further information is found in PCT Pub. No. WO 2025/019254 and 2025019297, each of which are fully incorporated by reference herein.

Described herein are methods and compositions related to biomarker identification. In various embodiments, the method includes determination of feature importance may be provided using survival models such as penalized Cox regression. In certain embodiments, specific genomic and epigenomic features are associated with increased or decreased risk.

In various embodiments, mutations in genes such as KRAS, STK11, and KEAP1 may be associated with increased risk. In various embodiments, certain epigenomic-derived features may be associated with reduced risk and correspond to methylation patterns affecting developmental transcription factors. In various embodiments, these features may be used as biomarkers for prognosis or therapeutic targeting.

Described herein are methods and compositions for clinical applications, which may include one or more of the following: Stratify patients into risk groups, predict disease progression, guide treatment selection, identify candidates for targeted therapies, and/or develop companion diagnostics. In various embodiments, are methods may be implemented on one or more computing systems including, one or more processors, memory storing executable instructions, interfaces for receiving biological and clinical data.

### Examples

### Example 1—Integrated Multi-Omic Clustering for Cancer Subtyping

In this example, a cohort of subjects diagnosed with advanced-stage cancer is analyzed using the disclosed framework. Clinical data, including treatment history and demographic variables, are obtained from electronic medical records. Genomic data comprising somatic mutation profiles are generated using next-generation sequencing, and epigenomic data comprising DNA methylation measurements are obtained from circulating nucleic acid samples.

The methylation data are processed to identify variable regions across the cohort, and principal component analysis is applied to generate a reduced set of latent epigenomic features. These features are combined with clinical and genomic features to form a unified feature matrix.

Multiple clustering algorithms, including k-means clustering and hierarchical clustering with different linkage criteria, are applied independently to the feature matrix. The resulting cluster assignments are aggregated to generate a consensus clustering using a co-association matrix.

Subjects are assigned to clusters based on the consensus clustering, and survival outcomes are evaluated using Kaplan-Meier analysis. Distinct clusters are observed to exhibit statistically significant differences in survival, thereby demonstrating that integrated multi-omic analysis enables identification of clinically meaningful subtypes.

### Example 2—Identification of Prognostic Epigenomic Features

In this example, the framework is used to identify prognostic features associated with clinical outcomes. Following clustering, a survival model is constructed using the combined feature set.

A penalized Cox proportional hazards model is applied to estimate the association between features and survival. The model identifies both genomic alterations and derived epigenomic features as significant predictors of outcome.

In particular, one or more latent epigenomic features derived from methylation data are found to be strongly associated with improved survival. Further analysis reveals that these features correspond to coordinated methylation patterns in genes associated with developmental regulation and cellular differentiation.

These findings demonstrate that epigenomic-derived features can serve as independent prognostic biomarkers and provide biological insights not captured by genomic data alone.

### Example 3 Exemplary Study

As an illustrative example of methods and compositions described herein, clinical data from 75,000 lines of therapy records of advanced lung cancer patients with an epigenomic methylation detection test result were integrated with genomic mutation data (3M variants) and targeted methylation data (top 1,000 variable regions) to predict clusters of progression on the following line of therapy. Features included clinical factors (therapy lines, regimens), binary mutation indicators (top-100 genes), and 20 principal components (PCs) derived from methylation data. Ensemble clustering using K-means and agglomerative methods identified patient subgroups. Survival was analyzed via Kaplan-Meier methods, and a penalized Cox proportional hazards model determined feature-level hazard ratios.

Four robust clusters emerged with distinct survival profiles (p<0.0001). Cox modeling (concordance=0.72) highlighted several prognostic markers: KRAS mutations (HR=1.24, p=0.01), STK11 mutations (HR=1.49, p<0.005), KEAP1 mutations (HR=1.33, p=0.01), and methylation PC5 (HR=0.03, p=0.01). Biological interpretation of PC5 revealed enrichment of hypermethylation in key developmental genes such as PAX6, SOX9, OTX2, WT1, and TBX20. This suggests epigenetic suppression of differentiation-related transcription factors contributes significantly to tumor aggressiveness.

### Example 4 Gene Association with Methylation Epigenomic Features

As shown in Table 1, the Top five genes associated with methylation principal component 5 (PC5), were ranked by absolute loading values from principal component analysis (PCA). PC5 was identified as a significant protective factor in the Cox proportional hazards model (hazard ratio<1), suggesting that methylation-driven changes in these developmental genes may play a crucial role in reducing tumor aggressiveness and improving patient survival. The listed genes (PAX6, SOX1, CLEC14A, OTX2, LOC112268136) represent potential epigenetic biomarkers for prognostic stratification in advanced lung cancer.

TABLE 1
Top Gene Associations

| | | absolute | | cumulative |
|---|---|---|---|---|
| gene name | loading | loading | proportion | proportion |
| SOX1-OT | 0.44 | 0.44 | 0.20 | 0.20 |
| CLEC14A | −0.40 | 0.40 | 0.16 | 0.36 |
| LOC112268136 | −0.40 | 0.40 | 0.16 | 0.36 |
| PAX6 | 0.27 | 0.27 | 0.08 | 0.43 |
| OTX2 | 0.18 | 0.18 | 0.03 | 0.47 |

### Example 5—Application to Treatment Stratification

In this example, the disclosed method is applied to guide treatment decisions for individual subjects. A trained model derived from a previously analyzed cohort is used to assign new subjects to one of several predefined clusters.

Upon receiving clinical, genomic, and epigenomic data for a new subject, the system processes the data to generate features consistent with those used in model training. The subject is then assigned to a cluster using the trained clustering framework.

Each cluster is associated with a distinct risk profile and therapeutic response pattern. Based on the assigned cluster, a treatment strategy is selected. For example, subjects assigned to higher-risk clusters may be recommended for more aggressive or combination therapies, while subjects assigned to lower-risk clusters may receive standard-of-care treatments.

This example illustrates how cluster-based subtyping can be used to personalize treatment decisions.

### Example 6—Use of Alternative Dimensionality Reduction Techniques

In this example, the epigenomic data are processed using a machine learning-based dimensionality reduction approach rather than principal component analysis.

Specifically, an autoencoder model is trained to learn a compressed representation of high-dimensional methylation data. The encoded latent features are extracted and used in place of principal components.

These latent features are combined with clinical and genomic features and subjected to ensemble clustering as described previously. The resulting clusters are evaluated with respect to clinical outcomes and are found to exhibit significant differences in survival.

This example demonstrates that alternative dimensionality reduction methods may be used to generate derived epigenomic features while still achieving meaningful disease subtyping.

### Example 7—Integration of Additional Modalities

In this example, additional biological data modalities are incorporated into the analysis. In addition to clinical, genomic, and epigenomic data, transcriptomic data representing gene expression levels are obtained for each subject.

The transcriptomic data are processed to generate features, which may include normalized expression values or derived components. These features are integrated with the existing feature set to form an expanded multi-omic representation.

Clustering is performed using the integrated dataset, and consensus clustering is applied to identify subgroups. The resulting clusters demonstrate enhanced separation with respect to clinical outcomes compared to clustering based on fewer data modalities.

This example illustrates that the disclosed framework can incorporate additional omic data types to improve predictive performance.

### Example 8—Application to Non-Cancer Disease

In this example, the disclosed methods are applied to a non-cancer disease characterized by heterogeneous clinical progression.

Clinical data, genomic variants, and epigenomic measurements are obtained from subjects diagnosed with the disease. Feature generation, dimensionality reduction, clustering, and outcome association are performed as described in previous examples.

The analysis identifies multiple subgroups with distinct progression patterns and risk profiles. Derived epigenomic features are found to be associated with disease severity and progression rate.

This example demonstrates that the disclosed framework is not limited to oncology and may be applied to other disease areas.

1. A computer-implemented method comprising:
receiving, for a plurality of subjects, at least two of: clinical data, genomic data, epigenomic data, transcriptomic data, and proteomic data;
generating a plurality of features including one or more derived epigenomic features obtained via dimensionality reduction;
applying a plurality of clustering algorithms to the features to generate candidate cluster assignments;
generating a consensus clustering based on the candidate cluster assignments;
assigning each subject to a cluster; and
associating the cluster with a clinical outcome.

2. The method of claim 1, comprising epigenomic data comprising DNA methylation data.

3. The method of claim 1, wherein the derived epigenomic features comprise principal components.

4. The method of claim 1, wherein the dimensionality reduction comprises principal component analysis.

5. The method of claim 1, wherein the dimensionality reduction comprises a machine learning model.

6. The method of claim 1, wherein the genomic data comprise mutation data.

7. The method of claim 6, wherein the mutation data comprise mutations in one or more cancer-associated genes.

8. The method of claim 7, wherein the genes comprise one or more of: PAX6, SOX9, OTX2, WT1, TBX20, KRAS, STK11, and KEAP1.

9. The method of claim 1, wherein the clustering algorithms comprise at least two different clustering methods.

10. The method of claim 9, wherein the clustering methods comprise k-means and hierarchical clustering.

11. The method of claim 9, wherein the clustering methods further comprise density-based clustering.

12. The method of claim 1, wherein generating the consensus clustering comprises constructing a matrix, optionally comprising co-association.

13. The method of claim 1, further comprising evaluating cluster stability.

14. The method of claim 1, wherein the clinical outcome comprises one or more of: progression-free survival, overall survival, progression, recurrence, adverse event, and time to next treatment.

15. A system comprising one or more processors and memory storing instructions that, when executed, cause the system to perform the method of claim 1.

16. A non-transitory computer-readable medium storing instructions that, when executed, cause a system to perform the method of claim 1.

17. A method of treating a subject comprising assigning the subject to a cluster using the method of claim 1 and selecting a therapy based on the cluster assignment.

18. The method of claim 1, further comprising fitting a survival model.

19. The method of claim 18, wherein the survival model comprises a Cox proportional hazards model.

20. The method of claim 18, further comprising identifying one or more prognostic biomarkers.

21. The method of claim 1, wherein the disease comprises cancer.

22. The method of claim 21, wherein the cancer comprises lung cancer.

23. The method of claim 1, wherein the epigenomic features correspond to methylation patterns associated with developmental genes.

24. The method of claim 23, wherein the genes comprise one or more of: PAX6, SOX9, OTX2, WT1, TBX20, KRAS, STK11, and KEAP1.

25. The method of claim 1, wherein the features further comprise clinical treatment history.

26. The method of claim 1, wherein the method comprises prediction of disease.

27. The method of claim 1, wherein the method comprises selection of a treatment.

28. The system of claim 15, wherein the system is implemented in a cloud computing environment.

29. The system of claim 15, further comprising an interface for receiving biological data.

30. The computer-readable medium of claim 16, wherein the instructions further cause identification of one or more prognostic biomarkers.

31. The method of claim 1, wherein the epigenomic data are derived from a blood sample.

32. The method of claim 1, wherein the epigenomic data are derived from a tissue sample.

33. The method of claim 1, wherein the features further comprise genomic, transcriptomic or proteomic data.

34. The method of claim 1, wherein assigning the subject to the cluster comprises assigning a new subject using a trained model.

35. The method of claim 17, wherein the therapy comprises a targeted therapy selected based on the cluster.
